(19) United States
(12) Patent Application Publication
(10) Pub. No.: US 2004/0229346 A1
(43) Pub. Date: Nov. 18, 2004

(54) Micro-particle array analysis system, micro-particle array kit, and chemical analysis method

(76) Inventors: Yoshinobu Kohara, Kokubunji (JP); Kazunori Okano, Shiki (JP); Hideyuki Noda, Kokubunji (JP)

Correspondence Address: MATTINGLY, STANGER & MALUR, P.C., SUITE 370, 1800 DIAGONAL ROAD, ALEXANDRIA, VA, 22314, US

(21) Appl. No.: 10/790,063

(22) Filed: Mar. 2, 2004

(30) Foreign Application Priority Data
May 12, 2003 (JP) 2003-132464

(51) Int. Cl. C12Q 1/68; C12M 1/34

(52) U.S. Cl. 435/287.2; 435/6

(57) ABSTRACT

There is provided means for analyzing organism-related molecules, dealing with multi item analysis, that are captured according to probe species, and for collecting according to the probe species. A magnetic micro-particle array is fixed with magnets that are configured with magnetic micro-particles in a capillary and with an array of glass beads to which DNA probes of different types from each other are immobilized. A syringe pump and a cross valve are operated to circulate a sample solution in the magnetic micro-particle array, which is reacted with probe DNAs on a glass bead with a probe. Subsequently, a washing solution is introduced to wash inside of the capillary. Next, respective beads are measured for fluorescence intensities. Furthermore a particular bead is collected based on results of fluorescence measurement. Target molecules captured on a surface of the collected bead may be separated by heat-denaturation, which then may be subjected to next analysis.

### FIELD OF THE INVENTION

The present invention relates to analysis of organism-related molecules, and specifically to analysis of nucleic acids, such as DNA and RNA, and proteins. Moreover, the present invention belongs to a field of a micro-particle array for analyzing organism-related molecules.

### BACKGROUND OF THE INVENTION

Microarrays for analyzing biological materials are frequently used especially for multi item analysis of DNA. A microarray is usually fabricated for many probes by being classified from one kind to another kind and by immobilized on a solid surface. The probe arrays include: a method in which an oligomer with a designed sequence is synthesized base by base in each of a large number of sectioned cells, using a lithography technology widely used in a photochemical reaction and a semiconductor industry (for example, nonpatent literature 1); and a method in which a probe solution is spotted one by one to each section (for example, nonpatent literature 2.) Any methods of fabrication of a DNA microarray requires much time and effort, resulting in disadvantages of high fabrication cost. In order to solve the disadvantages, a probe array using micro-particles, that is, a micro-particle array has been developed. That is, a micro-particle array in which probe-immobilized beads are fixed to an end of an optical fiber bundle (for example, nonpatent literature 3), and a probe array (beads array) in which probe-immobilized beads are arranged in a prescribed order in a capillary (for example, patent literature 1) are reported. Moreover, as a micro-particle array in which beads are not fixed, a method performing measurement with a flow cytometer simultaneously using two or more kinds of color-coded beads (for example, nonpatent literature 4) is reported.

On the other hand, as a technique for collecting organism-related molecules in a solution, a method utilizing micro-particles is used frequently. When nucleic acids in a solution needs to be collected, silica beads are mixed into the solution, and the beads are separated by centrifugal separation after absorption of the nucleic acids to a surface thereof, and then the nucleic acids are collected together with the silica beads. Moreover, a method is reported that in order to enable easy collection of micro-particles, magnetic micro-particles are used, and the magnetic micro-particles are separated and collected from a solution by disposing magnets close to the solution. For example, an automated equipment applying this process to extraction of nucleic acids is manufactured (for example, nonpatent literature 5).

[Nonpatent literature 1] Science, 251, and 767-773 (1991)

[Nonpatent literature 2] Science and 270, 467-470 (1995)

[Nonpatent literature 3] Science, 287, 451-452 (2000)

[Nonpatent literature 4] Clinical Chemistry, 43, 1749-1756 (1997)

[Nonpatent literature 5] Journal of Bioscience and Bioengineering, 91, 500-503 (2001)

[Patent literature 1] Official gazette of JP-A No. 243997/1999

### SUMMARY OF THE INVENTION

In conventional microarray methods, there are yet to be provided methods for taking out organism-related molecules captured on a microarray according to every probe classification, and analyzing in more detail. Since it is predicted that one organism-related molecule is not necessarily captured by one probe but two or more may be captured, it is extremely important to know the captured molecules in detail. Moreover, in the conventional micro-particle arrays, control of a position of given beads after arraying is difficult. Moreover, since collection methods of collecting organism-related molecules using the conventional micro-particle is by a batch operation, it can operate only one kind of bead once. Even when two or more kinds of different beads are used as an object of collection, identification and collection of beads from one type of bead to another is difficult, and therefore multi item analysis and a collection may not be performed successfully.

In consideration of the above-mentioned situation, the present invention aims at providing means for collecting and analyzing organism-related molecules captured according to probe classifications, dealing with multi item analysis. As means to attain the above-mentioned object, the present invention provides a micro-particle array analyzing system in which a micro-particle array having magnetic micro-particles arrayed and magnets for operating the magnetic micro-particles are combined together, a micro-particle array kit, and also a chemical-analysis method. In the micro-particle array, micro-particles with probes immobilized thereto are arrayed in channels formed in capillaries or chips, and an arraying order thereof is beforehand determined, in order to identify a type of the probe immobilized to the micro-particles. Micro-particles with no probe immobilized thereto may also be included in micro-particles in this micro-particle array. Moreover, it is necessarily required to use a magnetic micro-particle for a part of the micro-particles.

Moreover, the present invention comprises following steps: immobilizing micro-particles with probes immobilized thereto with a help of a magnet so as to disable flowing out from inside of a vessel; supplying a sample to a magnetic micro-particle array; capturing organism-related molecules included in the sample on the micro-particles; (3) operating the magnets to move a micro-particle corresponding to a target probe; and (4) collecting the moved micro-particle.

A micro-particle array analyzing system concerning the present invention comprises: a vessel holding magnetic micro-particles and/or non-magnetic micro-particles; introducing means for introducing a sample and a solution into the vessel; a position-control means disposed outside of the vessel for magnetically controlling a relative position of the magnetic micro-particles with respect to the vessel, wherein the magnetic micro-particles and/or non-magnetic micro-particles are included in a given sequence within the vessel. A non-magnetic micro-particle is a micro-particle that substantially does not have magnetism, and, for example, it has glass etc. as a raw material. The micro-particle array analyzing system may further comprise a detector for detecting a bond between a probe and an organism-related molecule included in the sample, and an analyzer for analyzing results of detection.

The position-control means may be magnet members movably provided outside of the vessel, and the magnet members may be members relatively movable with respect to the vessel. Moreover, it may also be electromagnets provided outside of the vessel, and the electromagnets may control capturing to the electromagnets, and dissociation from the electromagnets of the magnetic micro-particles depending on variation of magnetic field to be generated.

The vessel may have branched channels inside, the magnetic micro-particles and/or non-magnetic micro-particles may be included in one of the channels, and given magnetic micros-particles and/or non-magnetic micro-particles may be taken out from an opening end of other channels.

The present invention may further comprise: a transport mechanism for transporting, using a liquid flow or suchlike, particular molecules in the sample by collecting magnetic micros-particles and/or non-magnetic micro-particles that were taken out from the opening end of the vessel; and an electrophoresis apparatus or a mass spectroscope connected to the transport mechanism.

A micro-particle array kit concerning the present invention comprises: a vessel holding magnetic micro-particles and/or non-magnetic micro-particles; magnet members disposed outside of the vessel; and probes binding to a particular molecule and being immobilized to any one of positions inside the vessel, wherein the magnetic micro-particles and/or the non-magnetic micro-particles are included in a given sequences within the vessel. Moreover, the vessel may be a channel provided in a capillary or a substrate.

A chemical-analysis method concerning the present invention comprises the steps of: disposing a vessel including a probe specifically binding to a particular molecule and magnetic micro-particles and/or non-magnetic micro-particles arrayed in given sequence; introducing a sample and a solution including the particular molecule into the vessel; controlling a position of the magnetic micro-particles using magnet members disposed in an exterior of the vessel; and detecting a result of bonding between the particular molecule and the probe. Moreover, the method may further comprise a step for collecting the magnetic micro-particles and/or the non-magnetic micro-particles. In the case, the magnetic micro-particles may relatively be moved with respect to the vessel by motion of the magnet members relatively with respect to the vessel, and thereby the magnetic micro-particles or the non-magnetic micro-particles may be taken out from an opening end of the vessel by motion of the magnetic micro-particles, and then may be collected. Alternatively in this case, by controlling magnetic field of electromagnets using the electromagnets as a magnet member, capturing and dissociation of the given magnetic micro-particles by the electromagnets may be controlled, and thereby after being captured with the electromagnets the given magnetic micro-particles may be dissociated, and conveyed by a flow of a solution caused inside the vessel, and then may be taken out from the opening end of the vessel to be collected.

### BRIEF DESCRIPTION OF THE DRAWINGS

FIGS. 1A, 1B, 1C, and 1D are schematic diagrams showing a micro-particle array analyzing system based on one embodiment of the present invention, and a process of analysis thereof;

FIGS. 2A and 2B are schematic diagrams showing a magnetic micro-particle array and a magnet based on one embodiment of the present invention;

FIGS. 3A, 3B, 3C, and 3D are schematic diagrams showing operation steps of a magnetic micro-particle array and a magnet based on one embodiment of the present invention;

FIGS. 4A, 4B, 4C, 4D, 4E, 4F, and 4G are a schematic diagrams showing a magnetic micro-particle array based on one embodiment of the present invention, and using steps;

FIGS. 5A and 5B show a block diagram showing an analysis protocol for nucleic acid using electrophoresis, and a nucleic acid analysis system with a magnetic micro-particle array based on one embodiment of the present invention incorporated therein and an electropherogram; and

FIG. 6 is a block diagram showing an analysis protocol for protein using a mass spectroscope, and an analysis system for protein with a magnetic micro-particle array based on one embodiment of the present invention incorporated therein.

### DESCRIPTION OF THE PREFERRED EMBODIMENTS

Hereinafter, embodiments of the present invention will be described in detail with reference to figures.

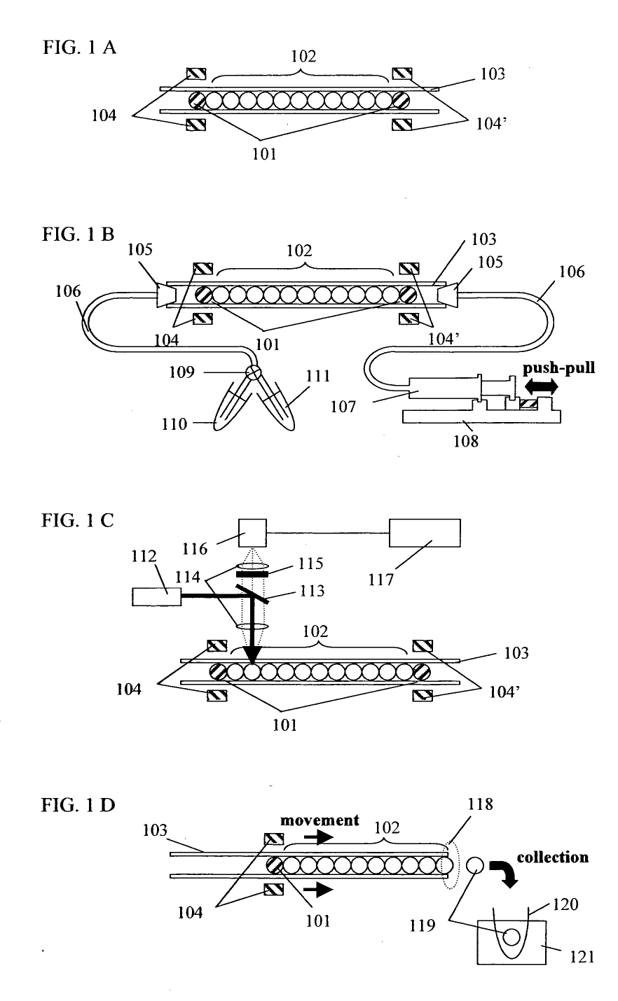

FIGS. 1A to 1D are diagrams for schematically showing a micro-particle array analyzing system and a process of analysis of a first embodiment of the present invention. FIG. 1A is an embodiment of a combination of a micro-particle array and magnets. In this embodiment, a micro-particle array is constituted in a row in a capillary 103. Magnetic micro-particles 101 are disposed in both ends of the array of the micro-particles, and glass beads 102 with probes are disposed inside thereof. DNA probes of different types are immobilized with respect to each glass beads 102, which are arrayed in a given sequence. Moreover, a configuration is adopted enabling identification of types of the probes according to an array order of the beads. Although various methods can be utilized for immobilization of the probe DNA to this glass beads, here, a method is adopted in which amino group is introduced into a glass bead with 3-amino propyl trimethoxy silane which is one of the silane coupling agents, then maleimido group is introduced into this amino group introduced beads with N-(11-maleimido undecanoxyloxy)succinimide, and subsequently a 5′-terminal thiol modified oligo DNA probe is immobilized thereto (for example, nonpatent literature 6). The magnetic micro-particles 101 and glass beads 102 have 100-micron diameter, respectively, and the capillary 103 has an inside diameter of 150 microns. Since diameters of the magnetic micro-particles 101 and the glass beads 102 with probes are larger than a half of the inner diameter of the capillary 103, an exchange in order is disabled between the magnetic micro-particles 101 and the glass beads 102 with probes, or between the glass beads 102. Previously reported fabrication methods may be used for fabrication of this micro-particle array (for example, patent literature 2, patent literature 3, patent literature 4). Magnets 104 and 104′ are disposed outside of the capillary to fix the magnetic micro-particles 101 inside the magnetic micro-particle array. In order to firmly fix the magnetic micro-particles 101, use of magnets having strong magnetism is effective, and, for example, rare earth neodymium magnets may suitably be used. Since the magnetic micro-particles 101 in both ends of the micro-particle array are fixed with respect to the capillary 103, disabling the glass beads 102 with the probe sandwiched between the magnetic micro-particles 101 to be taken out to exterior of the capillary 103. Although a method was conventionally adopted in which they are maintained inside the capillary by disposing obstructions, such as stainless steel wires on both sides of the micro-particle array, adoption of the above-mentioned configuration using the magnetic micro-particles and the magnets enables simple retain of beads inside of the capillary.

FIG. 1B is a schematic diagram showing a configuration of embodiment that operates a reaction to a combination of the micro-particle array and the magnets shown in FIG. 1A. In the case of reaction, a syringe 107 set to a syringe pump 108 is connected to an end of the micro-particle array through a connector 105 and an inner tube 106. Moreover, a sample vessel 110 and a washing solution vessel 111 through the connector 105, and the inner tube 106 and a cross valve 109 are connected to another end of the micro-particle array. The micro-particle array is disposed into a thermostat adjusted to suitable reaction temperatures throughout the reaction. In the case of detection of a target DNA in the sample solution, the target DNA is captured on beads using a hybridization reaction with probe DNAs on the glass beads 102 with probes, and in many cases temperatures in this case are usually set approximately 55 degrees C. The target DNA is fluorescence-labeled beforehand, and included in the sample vessel 110. Firstly, after operation of the cross valve 109 communicates the sample vessel 110 to the inner tube 106, the syringe 107 is drawn with a syringe pump 108, thereby the sample solution is vacuumed into the magnetic micro-particle array, and subsequently operation of the syringe pump 108 makes the sample solution circulate in order to accelerate a reaction between the probe DNA on the glass beads 102 with a probe. For example, after a reaction period for 10 minutes, the cross valve 109 is set so that the washing solution vessel 111 and the inner tube 106 might to be communicated, and then the sample solution is discharged from the magnetic micro-particle array, the washing solution is vacuumed with the syringe 107, and excessive sample solution remaining inside of the micro-particle array is washed off. At this time, most of the target DNA captured on the glass beads 102 with probes are left behind even after washing. Next, this target molecules are to be observed by fluorescence measurement.

FIG. 1C is a schematic diagram showing a configuration of embodiment of execution of fluorescence measurement after a reaction in a combination of the micro-particle array and the magnets as shown in FIG. 1B. A relative position of the micro-particle array, and the magnets 104 and 104′ are fixed, which does not vary a physical relationship between the magnetic micro-particles 101 inside the magnetic micro-particle array, and the glass beads 102 with probes, from a position before the reaction. Since background fluorescence may be emitted in the case of fluorescence measurement, a polyimide coating in a section of this capillary 103 used for magnetic micro-particle array where the micro-particle is arrayed is beforehand removed. The micro-particle array is scanned and fluorescence intensity from one bead to another bead is measured using a laser 112 as excitation light for fluorescence measurement. Firstly, the laser 112 is reflected by a dichroic mirror 113, and is cast to each of the glass beads 102 with a probe of the magnetic micro-particle array. Consequently, a fluorescent substance labeling the target DNA captured on beads is excited to emit an intrinsic fluorescence. Fluorescence generated on a surface of the beads is converged through a lens 114, is passed through an optical filter 115 corresponding to the fluorescence, and subsequently is converged to an acceptance surface of a photomultiplier tube 116. Signals from this photomultiplier tube 116 are analyzed by a personal computer 117, and an amount of fluorescence originating in each of the beads can be obtained. It is considered that this amount of fluorescence is correlated with an amount of the target DNA corresponding to the probe on the beads that existed in the sample.

FIG. 1D is a schematic diagram showing a configuration of embodiment in which currently a required bead is taken out, in a combination of the magnetic micro-particle array and the magnets shown in FIG. 1C, and a target DNA captured on the bead is collected. Although not shown in the Figure, the magnets can be moved in two or three dimensional directions by a magnets moving mechanism for controlling a position of the magnets. A particular bead selected based on measurement results in FIG. 1C is collected. When, for example, a result of the measurement requires collection of a second bead with the probe from a right end, the magnets 104′ are removed firstly and then the magnets 104 are moved to a right-hand side along with the magnetic micro-particle array. Thereby, the bead with a probe can be forced out in a sequential order following the magnetic micro-particle from the opening 118 of the capillary 103. After a first bead from a right end is forced out and collected in the vessel for beads to be abandoned, when forcing out is further performed, a second bead 119 is forced out from the opening, which is collected in the vessel 120 for collection. Collection operation includes two methods: a method in which operation of the magnets 104 is performed while observing the magnetic micro-particle array under a microscope; and a method in which a moving distance of the magnets 104 is beforehand calculated, and the magnets 104 are moved based on the calculated result. A target DNA captured by the probe exists on a surface of the collected bead 119. The vessel 120 for collection is placed on a heat block 121, heated about 5 minutes at 94 degrees C., and thermally denaturated, and thereby the target DNA may easily be collected into a solution. Although an embodiment is illustrated here in which the target DNA is denaturated by thermal denaturation using a heat block to be collected, a method may also be adopted in which a collected bead is heated by laser irradiation in a solution, and furthermore a method also may be adopted in which a collected bead is denatured with alkali using about 0.1M sodium hydroxide solution.

Although, in this embodiment, only one of the magnetic micro-particle exists in each of both ends of the micro-particle array, respectively, a plurality of corresponding magnetic micro-particles may exist in both ends, and further they may exist in positions other than the end positions. Moreover, magnetic micro-particles with probes may be used instead of glass beads. Moreover, all of the micro-particles in the array may be magnetic micro-particles with probes.

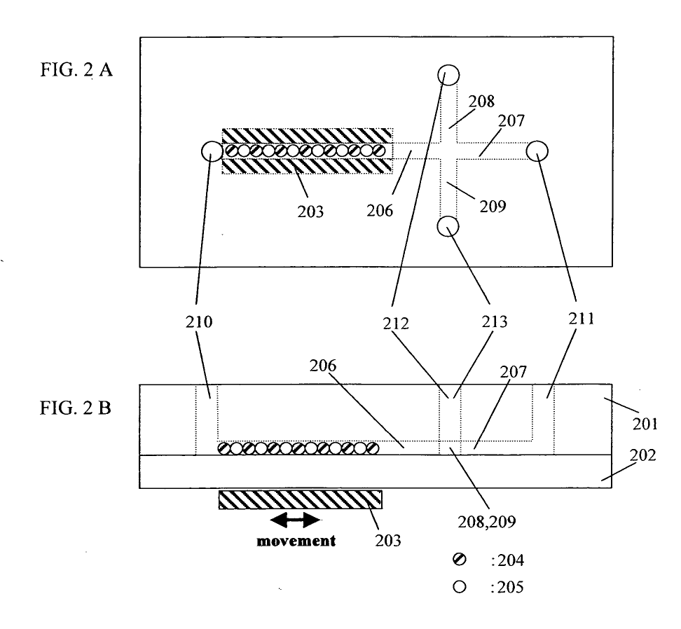

FIGS. 2A and 2B show schematically an outline of a relationship between a micro-particle array and a magnet of second embodiment of the present invention. FIG. 2A is an overhead schematic diagram of a micro-particle array and a magnet; and FIG. 2B is a side schematic diagram of the magnetic micro-particle array and the magnet. This magnetic micro-particle array differs from the first embodiment, and is configured inside a chip. A micro-particle array that is arrayed in a channel 206 inside the chip is operated with an external magnet 203. This chip has a structure where a resin section 201 made of PDMS (polydimethylsiloxane) as a material and a slide glass 202 are attached together. Channels 206 to 209 having a form of a cross joint are formed in a side where the PDMS section 201 of the chip contacts the slide glass, and a magnetic micro-particle array is configured in one channel 206 of the channels. In order to manufacture the channels 206 to 209 in this PDMS resin section 201, for example, there is used a mold manufactured by a method (for example, nonpatent literature 7) of using a technique of photo lithography often used in a manufacturing process of semiconductors. A light is irradiated, through a mask having a configuration of this channel reflected thereto, on a silicon substrate spin coated with SU-8 or one of photoresists, and thus the mold is manufactured. A liquefied mixture of a PDMS and a solidification catalyst is poured on this mold, heated at 200 degrees C. for about 1 hour, then removed from the mold, and thus a PDMS section 201 of the chip may be obtained. Through holes are given at tips of each channel by punching, and thereby piping openings 210 to 213 communicating to external pipings through connectors are provided. After a bonding surface of this PDMS section 201 is irradiated with oxygen plasma, it is attached on the slide glass 202. In this embodiment, magnetic micro-particles 204 and glass beads 205 with probes are arrayed in alternately given sequences, and arrangement between beads 205 with probes is beforehand decided based on probe species on the beads. This magnetic micro-particle array may also be fabricated by a same fabrication method as in the first embodiment. Respective channels 206 to 209 have shapes of 130-micron square, and magnetic micro-particles 204 and glass beads 205 with probes have 100-micron diameter, which disables exchange in order between the magnetic micro-particles 204 and the glass beads 205 with probes. This micro-particle array was used in order to specifically capture fluorescence labeled target DNAs in a sample solution and to collect them, using a DNA probe as a probe as in the first embodiment. The magnetic micro-particles 204 and the glass beads 205 with probes which were arrayed in the channel 206 can be operated by moving the magnets 203 along the channel 206. During reaction and washing, in order to keep reaction temperatures constant, the chip and the magnet 203 are held on a heat-regulated plate where temperatures are controlled. This chip has a dimension of 25 mm×75 mm, which is same as that of a slide glass by JIS specification. The slide glass has a thickness of 1 mm, the PDMS section has a thickness of 2 mm, and these sum total is enough thin to give 3 mm. Since this small thickness enables fluorescence measurement using an existing DNA chip scanner, the method was adopted in this embodiment.

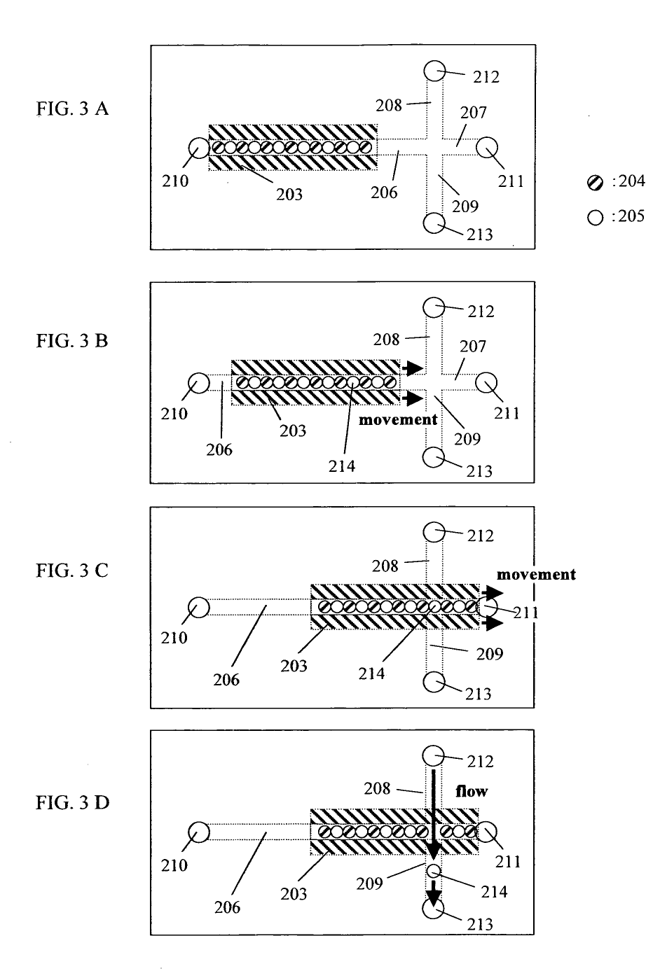

FIG. 3A to 3D show a schematically step for taking out the beads after reaction to the micro-particle array of the second embodiment of the present invention. They all are overhead schematic diagrams of the chip, and the magnets 203 are disposed through the slide glass 202. FIG. 3A is a schematic diagram showing a physical relationship of the magnetic micro-particle array chip and the magnets 203 in the reaction and washing process. The magnetic micro-particles 204 and the glass beads 205 with probes are fixed by the magnets 203 disposed beneath the chip. Circulation of a sample solution and washing after reaction with the sample are performed using piping openings 210 and 211 among the piping openings of the chip. A syringe connected to a syringe pump is connected to the piping opening 211 through a connector and a tube as in FIG. 1 showing the first embodiment. Moreover, a sample vessel and a washing solution vessel through a connector, a tube, and a cross valve are connected to the piping opening 210. According to the procedure described in the first embodiment, reaction and washing of the sample are performed after these connecting operations. FIGS. 3B and 3C are schematic diagrams in which the magnetic micro-particles 204 and the glass beads 205 with probes are moved by the movement of the magnet 203. A bead 214 with a probe to be a target of collection is moved to a point where four channels 206 to 209 intersect together to give a shape of a cross. Although the magnet 203 is moved in this Figure, a same effect may also be obtained by fixing a position of the magnet and by moving the chip. FIG. 3D is a schematic diagram in which a glass bead 214 with a probe to be a target of collection is collected through the channel 209. Here, the target glass bead is moved by a force of a flow of a solution. A pump is connected to a piping opening 212 and a solution is flown through the channels 208 and 209 intersecting the channels 206 and 207 where the magnetic micro-particles 204 and the glass beads 205 with probes are arrayed. Here, pure water is used as a solution. A flow of solution moves the glass bead 214 to be a target of collection that is held with a frictional force, which is thus collected from the piping opening 213.

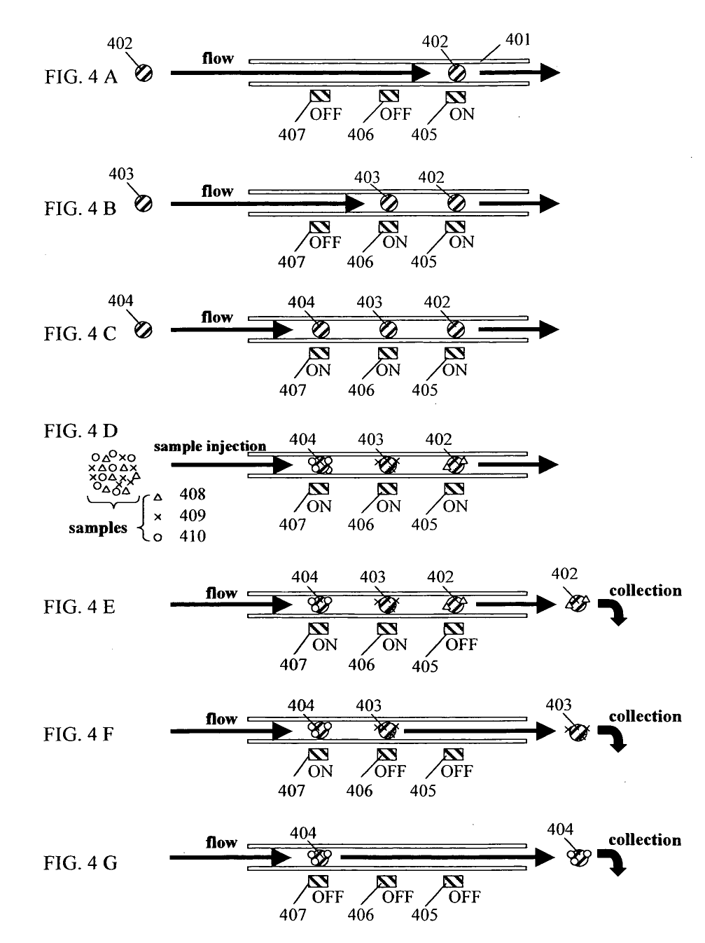

FIG. 4A to 4G are schematic diagrams showing the magnetic micro-particle array, and an outline of the operational method of the magnetic micro-particle array of a third embodiment of the present invention. In this embodiment, a magnetic micro-particle array having two or more kinds of magnetic micro-particles with probes arrayed in a given order therein is prepared. Each target in samples is captured by each magnetic micro-particle with a probe, and is individually collected, respectively. Electromagnets 405 to 407 of a number equal to types of probes are disposed in exterior of a capillary 401, and magnetic micro-particles are operated by these electromagnets 405 to 407 and by a flow of a solution in the capillary 401. Firstly, a magnetic micro-particle 402 with a first probe immobilized thereto is poured into the capillary 401. In this case, an electromagnet 405 in a most distant position from an entrance of pouring in is turned on, remaining electromagnets 406 to 407 are turned off, and thereby the magnetic micro-particle 402 is fixed to a position of the endmost electromagnet 401. Next, when a magnetic micro-particle 403 having a second probe immobilized thereto is poured in, a second electromagnet 405 from an end is turned on, and thereby the magnetic micro-particle 403 is fixed to a position currently fixed. By repeating such steps, magnetic micro-particles with different probes immobilized thereto may be fixed in the capillary 401 in a prescribed order, and thus a magnetic micro-particle array with given sequences may be configured. After the magnetic micro-particle array is configured, all electromagnets 405 to 407 are kept at on. After a sample solution is circulated to this magnetic micro-particle array to perform reactions, and target molecules are captured by each magnetic micro-particle with a probe, washing is performed. This reaction and washing steps are performed by a same method as in the first embodiment. In this embodiment, fluorescence measurement of targets is not performed but each of the magnetic micro-particles is immediately collected separately. Firstly, a solution is poured into the capillary 401, and by turning off a first electromagnet 405 from a downstream of a flow, a first magnetic micro-particle 402 with a probe is collected with target molecules. Next, the second magnetic micro-particles 403 with a probe are collected with target molecules by turning off a second electromagnet 406 from a downstream in the flow. By repeating same procedures henceforth, that is, by repeating operations of turning off an electromagnet in state of on in a most downstream side of the flow, the magnetic micro-particles 402 to 404 having specific target molecules 408 to 410 captured thereto, respectively, can be independently taken out.

Although an embodiment in which a probe is immobilized to a magnetic micro-particle is shown in this embodiment, same results may be obtained also with a combination of a magnetic micro-particle without a probe, and a glass bead with a probe. By a procedure in which a magnetic micro-particle is firstly fixed in a channel with an electromagnet and subsequently a glass bead is poured in, the glass bead can be kept in a given position by the fixed magnetic micro-particle. Moreover, in the case of collection, as in the above-mentioned case, when the electromagnet is turned off, the glass bead capturing specific target molecules can be independently taken out.

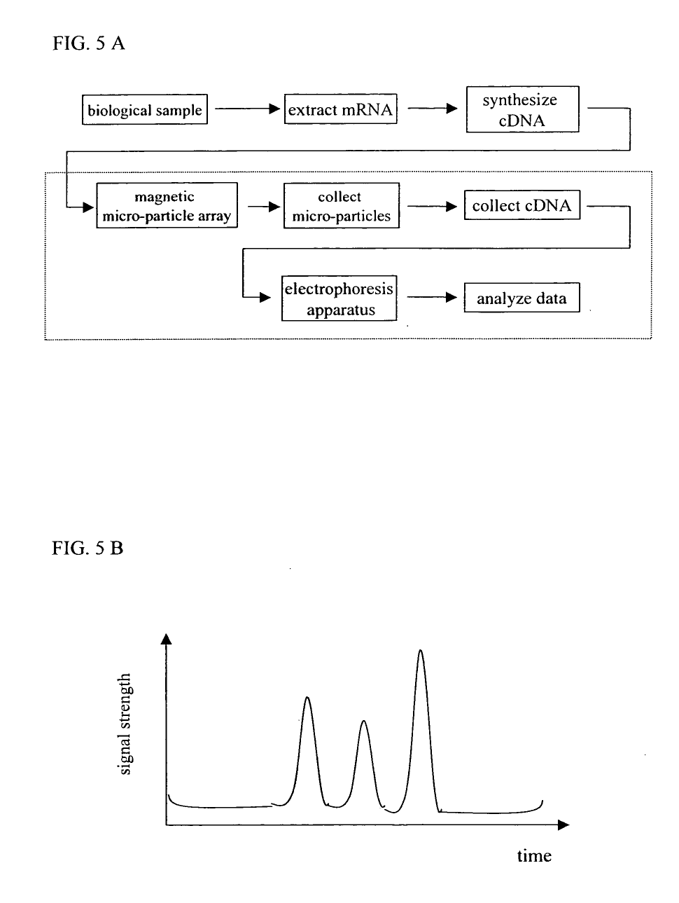

FIG. 5A is a block diagram of nucleic acid analysis system by electrophoresis in which a magnetic micro-particle array based on one embodiment of the present invention is incorporated. In the figure, a section surrounded by dotted line is a section where a magnetic micro-particle array based on one embodiment of the present invention and operations therefore are built in. Here, assumed is a system in which an expression profile measurement of mRNA of multiple item inspection is performed by the magnetic micro-particle array, micro-particles with probes are collected from inside of the micro-particle array based on fluorescence detection results, target DNAs captured by the collected micro-particle with a probe may be separated from the probe with heat, and a length thereof is analyzed with electrophoresis apparatus. Firstly, mRNA is extracted from target tissue of examination, here 1 mL of whole blood of human beings, and then a cDNA group that may be detected with fluorescence is synthesized using a reverse transcriptase and a fluorescence labeled dNTP. Thus prepared sample can be introduced into the magnetic micro-particle array to enable observation with fluorescence, and thereby a multiple item expression profile analysis can be performed. Here, a micro-particle array configured in a chip made of a PDMS-slide glass as in the second embodiment was used. When micro-particles with probes showing a behavior with a significant fluorescence intensity is found as a result of expression frequency analysis, the micro-particles can be collected using the above-mentioned method. Since a splicing variant of p53 gene was set as an observable target here, a probe which has a sequence complementary to each exon of the p53 is immobilized to a glass bead to fabricate a magnetic micro-particle array. A cDNA captured on the collected micro-particle can be separated from the micro-particle by thermal denaturation or alkali denaturation. The cDNA solution 10 μL of a total amount of separated 50 μL is introduced into a 10× loading buffer for electrophoreses 1 μL, and then a length is eventually analyzed with a capillary-electrophoresis apparatus. Inside of capillary of 30 cm is filled with 4% of poly linear acryl amide to be used. Loading of sample was performed by application with a voltage of 0.75 kV for 10 seconds, and a voltage of 1.5 kV was applied in electrophoresis. A cDNA collected from the micro-particle to which a probe complementary to exon 8 of the p53 was immobilized is applied to electrophoresis, and as a result, three bands were observed as shown in FIG. 5B. Separately collection of micro-particles from the micro-particle array may realize an individual collection having a little contamination between probes.

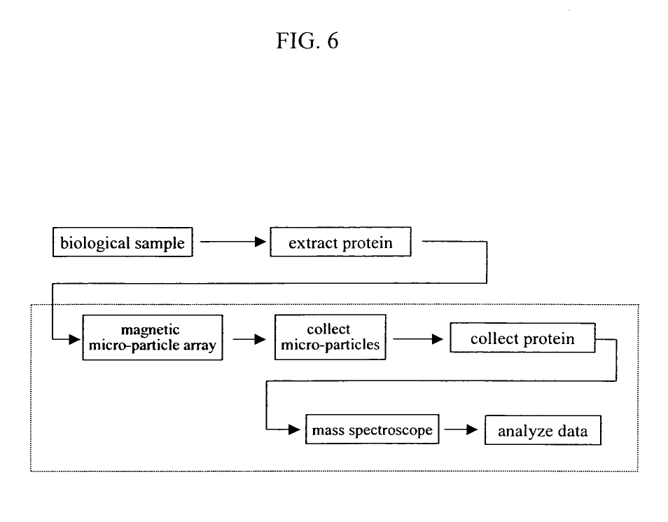

FIG. 6 is a block diagram of protein analysis system by mass spectroscope according to one embodiment of the present invention in which a magnetic micro-particle array is incorporated. In the Figure, a section surrounded by dotted line is a section where a magnetic micro-particle array based on one embodiment of the present invention and operations therefore are built in. Here, assumed is a system in which proteins dealing with multiple item analysis are captured by a magnetic micro-particle array to which a double strand DNA with different sequence is immobilized as a probe, micro-particles after captured are collected one by one, a protein group captured by the collected micro-particle is denaturalized, and separated, and then molecular weights are analyzed using a mass spectroscope. In this embodiment, analysis was performed for the purpose of DNA-binding protein with DNA-binding ability corresponding to each probe sequence being captured, and of obtaining molecular weight information thereof. Firstly, a protein group is extracted from tissues or biological species as targets whose DNA-binding proteins are to be examined, here 100 ml of cultivated yeast. This protein group is dissolved in pH7-Tris buffer solution so as to give a concentration of about 1 mg/mL. Thus prepared sample is introduced into a magnetic micro-particle array, and a capturing reaction to probes on each micro-particle is accelerated by a circulating movement in the micro-particle array of the sample. Here, a micro-particle array configured in a capillary was used as in the first embodiment. Each micro-particle may be collected in a sequential order according to the above-mentioned method. DNA-binding proteins captured on the collected micro-particles may be thermally denaturated at 94 degrees C. and for 30 minutes in pure water 10 mL to be separated from the micro-particle. The separated protein solutions 1 mL of a total amount 10 mL was mixed with a matrix, a molecular weight distribution thereof might be measured using a matrix-assisted laser desorption ionization time of flight mass spectrometer. Although double strand DNAs are immobilized as a probe in this embodiment, a system may be adopted in which organism-related low molecules and proteins are immobilized as a probe.

[Nonpatent literature 6] Nucleic Acids Research, 30, and e87 (2002)

[Nonpatent literature 7] Electrophoresis, 22, 328-333 (2001)

[Patent literature 2] Official gazette of JP-A No. 243997/1999

[Patent literature 3] Official gazette of JP-A No. 346842/2000

[Patent literature 4] Official gazette of JP-A No. 117487/2002

By controlling positions of magnetic micro-particles in a magnetic micro-particle array, organism-related molecules captured by a probe may be analyzed while the probe immobilized to the micro-particle being identified, which enables multi item analysis and collection based on probe species.

Moreover, means may be provided for analyzing organism-related molecules captured by the probe, and for collecting them based on the probe species for another kind of analysis.

Furthermore a practical system may be provided for capturing and analyzing organism-related molecules at low cost.

What is claimed is:

1. A micro-particle array analyzing system comprising:
a vessel holding at least a magnetic micro-particle and/or at least a non-magnetic micro-particle;
introducing means for introducing a sample and a solution into the vessel; and
a position-control means disposed outside of the vessel for magnetically controlling a relative position of the magnetic micro-particle with respect to the vessel,
wherein the magnetic micro-particle and/or non-magnetic micro-particle are included in a given sequence within the vessel.

2. The micro-particle array analyzing system according to claim 1, wherein the non-magnetic micro-particle has a probe immobilized to a surface thereof, and is included in the vessel to be sandwiched between the magnetic micro-particles.

3. The micro-particle array analyzing system according to claim 1, wherein a plurality of magnetic micro-particles are used and at least one of the magnetic micro-particles has a probe immobilized to a surface thereof.

4. The micro-particle array analyzing system according to claim 2, further comprising:
a detector for detecting a bond between the probe and organism-related molecules included in the sample; and
an analyzer for analyzing results of detection.

5. The micro-particle array analyzing system according to claim 1, wherein the position-control means is a magnet member movably provided outside of the vessel.

6. The micro-particle array analyzing system according to claim 1, wherein the position-control means is an electromagnet provided outside of the vessel, and the electromagnet controls capturing to the electromagnet, and dissociation from the electromagnet of the magnetic micro-particle depending on variation of magnetic field to be generated.

7. The micro-particle array analyzing system according to claim 1, wherein the vessel has branched channels inside, the magnetic micro-particle and/or the non-magnetic micro-particle are included in one of the branched channels, and the given magnetic micros-particle and/or the given non-magnetic micro-particle are taken out from an opening end of one of other channels.

8. The micro-particle array analyzing system according to claim 1, further comprising:
a transport mechanism for transporting particular molecules in a sample by collecting the magnetic micro-particle and/or the non-magnetic micro-particle being taken out from an opening end of the vessel; and
an electrophoresis apparatus connected to the transport mechanism.

9. The micro-particle array analyzing system according to claim 1, further comprising:
a transport mechanism for transporting particular molecules in a sample by collecting the magnetic micro-particle and/or the non-magnetic micro-particle being taken out from an opening end of the vessel; and
a mass spectroscope connected to the transport mechanism.

10. A micro-particle array kit comprising:
a vessel holding at least a magnetic micro-particle and/or at least a non-magnetic micro-particle;
a magnet member disposed outside of the vessel; and
a probe binding to a particular molecule and being immobilized to any one of positions inside the vessel,
wherein the magnetic micro-particle and/or non-magnetic micro-particle are included in a given sequence within the vessel.

11. The micro-particle array kit according to claim 10, wherein the probe is immobilized to the non-magnetic micro-particle.

12. The micro-particle array kit according to claim 10, wherein the probe is immobilized to the magnetic micro-particle.

13. The micro-particle array kit according to claim 10, wherein the vessel is a channel provided in a capillary or a substrate.

14. A chemical-analysis method comprising the steps of:
disposing a vessel including a probe specifically binding to particular molecules and at least a magnetic micro-particle and/or at least a non-magnetic micro-particle arrayed in a given sequence;
introducing a sample and a solution including the particular molecules into the vessel;
controlling a position of the magnetic micro-particle using a magnet member disposed in an exterior of the vessel; and
detecting a result of bonding between the particular molecules and the probe.

15. The chemical-analysis method according to claim 14, wherein the probe is bonded to the magnetic micro-particles.

16. The chemical-analysis method according to claim 14, wherein the probe is bonded to the non-magnetic micro-particles, and in a step of being included in the vessel the non-magnetic micro-particle is included in the vessel being sandwiched between the magnetic micro-particles.

17. The chemical-analysis method according to claim 16, further comprising a step of:
collecting the magnetic micro-particle and/or the non-magnetic micro-particle, wherein
in a step of controlling a position of the magnetic micro-particle, the magnetic micro-particle is relatively moved with respect to the vessel by motion of the magnet member relatively with respect to the vessel, and
in a step of collecting, the magnetic micro-particle or the non-magnetic micro-particle are taken out from an opening end of the vessel by motion of the magnetic micro-particle, and then collected.

18. The chemical-analysis method according to claim 15, further comprising a step of:
collecting the magnetic micro-particle and/or the non-magnetic micro-particle, wherein
in a step of controlling a position of the magnetic micro-particle, capturing and dissociation of the given magnetic micros-particle by an electromagnet is controlled by controlling magnetic field of the electromagnet using the electromagnet as a magnet member, and
in a step of collecting, after being captured with the electromagnet the given magnetic micros-particle is dissociated, and conveyed by a flow of a solution caused inside the vessel, and then is taken out from an opening end of the vessel to be collected.
